(19) United States
(12) Patent Application Publication
(10) Pub. No.: US 2017/0056383 A1
(43) Pub. Date: Mar. 2, 2017

(54) TOPICAL HERPES PAIN TREATMENT AND COMPOSITION

(71) Applicant: Julian Chisolm Brantley, III, Rocky Mount, NC (US)

(72) Inventor: Julian Chisolm Brantley, III, Rocky Mount, NC (US)

(21) Appl. No.: 14/840,606

(22) Filed: Aug. 31, 2015

(51) Int. Cl. A61K 31/445; A61K 9/00; A61F 13/40; A61K 31/167

(52) U.S. Cl. CPC A61K 31/445; A61K 31/167; A61K 9/0014; A61M 35/006

(57) ABSTRACT

The present invention is the treatment of Herpes simplex virus lesion pain via application of a product containing a quick acting and long acting topical anesthetic.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

Field of the Invention

The present invention relates to a method and composition for the treatment of pain associated with lesions cause by the Herpes virus. In particular, the present invention relates to a combination of a short or intermediate acting and long acting topical local anesthetic applied to treat the pain associated with the Herpes Simplex Virus lesions.

Description of Related Art

Herpes Simplex Virus (HSV) creates inflammation, most frequently around the mouth and genitals, with a tendency to break open at some point and create painful open lesions with exposed nerve endings. The type of HSV associated with lesions of the mouth and facial region is Type I, while Type II is most commonly associated with lesions of the genital region.

Because both the mouth and genitals are exposed to a great deal of activity, movement and contact, these activities cause constant, unbearable irritation for some people dealing with lesions. Treatments for HSV are of limited efficacy. Since there is no cure at this time, treatments center around pain management. The classic treatment is to apply benzocaine to the lesion, but, with an active working time of only about 15 minutes, this is not the best treatment for the pain management in most people. Short acting topical anesthetics, however, have the advantage of working almost instantly, providing relief as soon as they are applied, even if the relief does not last very long. Intermediate acting local anesthetics have a duration of action of about 45 minutes and a slightly slower onset of action (˜45 seconds).

Long acting topical anesthetics, those lasting 6 hours or more, are effective for extended time periods, but take a longer time to begin providing relief. There is currently a great need to have a more effective product for the treatment of pain associated with Herpes lesions.

### BRIEF SUMMARY OF THE INVENTION

The present invention relates to the discovery that applying a combination of a short acting or intermediate and a long acting topical anesthetic solves the problems above in an easy to use product. Upon application to a lesion, relief is immediate and is characterized by decreases in pain and swelling, and more rapid healing compared to an untreated lesion and relief lasts for hours.

Accordingly, in one embodiment, the present invention relates to a composition for the treatment of the pain associated with Herpes simplex lesions comprising:
a) a short or intermediate acting local anesthetic between about 5% to about 95% w/w of the total anesthetics in the composition;
b) a long acting local anesthetic of between about 5% to about 95% w/w of the total anesthetics in the composition; and
c) the composition formulated as a liquid for application to human tissue.

In another embodiment, it relates to a method of treating the pain associated with a Herpes simplex lesion comprising:
a) selecting a composition comprising;
   i. a short or intermediate acting local anesthetic between about 5% to about 95% w/w of the total anesthetics in the composition;
   ii. a long acting local anesthetic of between about 5% to about 95% w/w of the total anesthetics in the composition; and
   iii. the composition formulated as a liquid for application to human tissue;
b) applying the composition to the lesion.

### BRIEF DESCRIPTION OF THE DRAWINGS

FIG. 1 is a swab impregnated with the composition of the present invention.

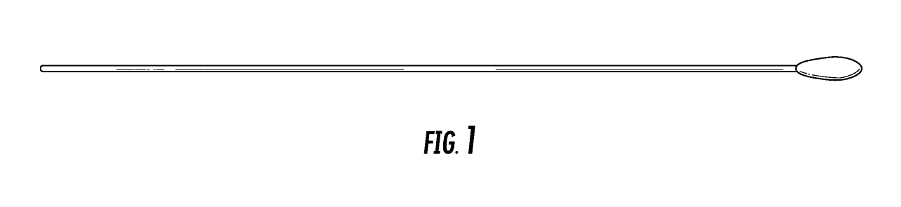

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “composition” refers to a liquid or gelled composition containing both a short or intermediate acting and long acting local anesthetics. Such liquid and gelled compositions are within the skill in the art. The composition can be made by combining each local anesthetic in its own liquid or gel, or placing the local anesthetics in a single liquid or gel. Such techniques are within the skill in the art. Examples of carriers that are liquid or gel can be seen below, but are merely exemplary.

As used herein, the phrase “local anesthetic” refers to a composition which, when topically applied in a liquid or gelled formulation, such as application to a Herpes lesion, the composition causes local analgesia where applied but does not cause undue systemic absorption or effects. Typically, short or intermediate acting local anesthetics have a very rapid and, in some cases, almost instant onset of action, but have a relatively short period of duration i.e. an hour or less. Short or intermediate acting anesthetics include Benzocaine, chloroprocaine, hexylcaine, procaine, hydroxyprocaine, oxybuprocaine, proparacaine, tetracaine, lidocaine, and mepivacaine. Long acting anesthetics, on the other hand, have a slow onset of action, taking minutes to achieve analgesia, but can last 6 hours or more. Long term acting anesthetics include Articaine, bupivacaine, cenchocaine, etidocaine, levobupivacaine, prilocaine, ropivacaine and trimecaine The list of compositions is not intended to be limiting but, rather, only to give examples so that one skilled in the art can understand short and long acting anesthetics. Each of the short/intermediate and long acting local anesthetics can be about 5% to about 95% on a weight/weight (w/w/) basis of the total anesthetics in the composition. In one embodiment, each is between 40% and 60% w/w. The total amount of anesthetic can be about 0.1% to 10% of the total composition, with 2% representing 2 g/100 cc.

As used herein, the phrase “formulated for application to human tissue” refers to a liquid or gelled composition which is pharmaceutically compatible with application to broken skin or tissue, such as is the case with a Herpes lesion. Such formulations are within the skill in the art in view of the teaching herein. As used herein and in the claims, the term “liquid” shall include gels.

As used herein, the term “applicator” refers to a device for applying a small amount of a liquid or gel composition of the present invention. Droppers, swabs, pads or the like, or anything known in the art for applying a small quantity of about a drop to the applicator, and then touching the lesion with the application to apply a small amount to the lesion. The applicator can be sealed individually or as a group for use. For example, where using a swab, the composition can be applied to the swab and the swab sealed in a plastic disposable container.

As used herein, the phrase “method of treating pain” refers to the liquid composition of the present invention applied to a Herpes simplex lesion. In one embodiment, the composition is applied to an applicator and the applicator used to apply the composition to the lesion. For example, a drop of the composition is applied to a swab or cotton tipped swab, or the like, and the swab utilized to apply the composition by rubbing the swab over the lesion.

The composition of the present invention can be added to and delivered in a conventional pharmaceutical carrier for topical application to a human, especially the facial or genital lesions of HSV. For example, the composition is mixed with one or more suitable excipients to maximize delivery of the anesthetic. In some embodiments, the anesthetics are incorporated into a cream, gel, lotion, ointment, foam, suppository, or a spray, using methods known in the art. Suitable excipients include emulsifiers, organogelators and emollients. Emulsifiers include polyethylene glycol stearate, a glycol stearate, a glyceryl stearate, cetearyl alcohol and ceteareth 20, methylcellulose, Cetomacrogol 1000, and lecithin. Suitable organogelators include 4-tertbutyl-1-aryl cyclohecanols derivatives, polymeric (e.g. poly(ethylene glycol), polycarbonate, polyesters, and poly(alkylene), Gemini gelators (e.g. N-lauroyl-L-lysine ethyl ester), Boc-Ala(1)-Aib(2)-β-Ala(3)-OMe (synthetic tripeptide), and low molecular weight gelators (e.g. fatty acids and n-alkanes). Suitable emollients include cetostearyl alcohol, cetyl alcohol, isopropyl palmitate, caprylic/capric triglyceride, PPG-2 myristyl ether propionate, dimethicone, methicone, petrolatum, lanolin, and mineral oil.

If desired other additives, including surfactants, penetration enhancers, preservatives, viscosity modifiers, and emulsion stabilizers, may be included in the anesthetic compositions. Suitable surfactants include sodium lauryl sulfate, cetostearyl alcohol, ceteareth 12, ceteareth 20, cetearyl alcohol, Cetomacrogol 1000, stearic acid, and poloxamer. Suitable penetration enhancers include propylene glycol. Suitable preservatives include methylparaben, propylparaben, ethylhexylglycerin, phenoxyethanol, chlorocresol, potassium sorbate, sorbic acid, bronopol, methychloroisoihiazolinone, and methylisothiazolinone. Suitable viscosity modifiers include carboxymethylcellulose, carboxyethylcellulose, acrylate crosspolymer, and carbomer. Suitable emulsion stabilizers include xanthan gum, glyceryl stearate, and carbomer. If desired, other additives may be included to modify the color or aroma of the topical compositions described herein.

In one embodiment, the anesthetics are incorporated to a final weight percentage between 15% and 90% of a mixture of the anesthetics and carrier.

The resulting topical composition can be used to treat many conditions in which cutaneous nerves are involved, including acute pain, pain and itching associated with mosquito bites, wasp and bee stings, spider bites and burns; as well as the lesions of HSV.

### Examples of Compositions and Method of Application

One method, for example, would be having a foil wrapped cotton tipped applicator and a second isolated compartment that contains a mixture of a long-acting local anesthetic and an intermediate acting anesthetic (i.e. ½ 2% xylocaine and ½ 0.75% Bupivicaine). This second compartment would contain 0.25 cc of this solution in a viscous base.

The user would take the applicator and saturate the tip with the viscous solution and then place it directly on the herpetic ulcer. The user would hold this applicator on the lesion for 30 seconds and anesthesia would be obtained for 4-6 hours.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A composition for the treatment of the pain associated with Herpes simplex lesions comprising:
a) a short or intermediate acting local anesthetic between about 5% to about 95% w/w of the total anesthetics in the composition;
b) a long acting local anesthetic of between about 5% to about 95% w/w of the total anesthetics in the composition; and
c) the composition formulated as a liquid for application to human tissue.

2. The composition according to claim 1 which further comprises an applicator for applying the liquid composition to a Herpes simplex lesion.

3. The composition according to claim 2 wherein the application is a swab.

4. The composition according to claim 3 wherein the liquid is pre-applied to the swab.

5. The composition according to claim 2 wherein the composition is in a separate container for application with the applicator.

6. The composition according to claim 1 wherein the short or intermediate acting local anesthetics are selected from the group consisting of Benzocaine, chloroprocaine, hexylcaine, procaine, hydroxyprocaine, oxybuprocaine, proparacaine, tetracaine, lidocaine, and mepivacaine.

7. The composition according to claim 1 wherein the long acting local anesthetic is selected from the group consisting of Articaine, bupivacaine, cenchocaine, etidocaine, levobupivacaine, prilocaine, ropivacaine and trimecaine.

8. The composition according to claim 1 wherein the short or intermediate acting anesthetic is lidocaine and the long acting local anesthetic is bupivacaine.

9. The composition according to claim 8 wherein the anesthetics are present each from about 40% to 60% w/w as a percent of the anesthetics.

10. A method of treating the pain associated with a Herpes simplex lesion comprising:
a) selecting a composition comprising;
i. a short or intermediate acting local anesthetic between about 5 to about 95% w/w of the total anesthetics in the composition;
ii. a long acting local anesthetic of between about 5 to about 95% w/w of the total anesthetics in the composition; and
iii. the composition formulated as a liquid for application to human tissue;
b) applying the composition to the lesion.

11. The method according to claim 10 wherein the liquid is applied to an applicator and the applicator used to apply the liquid to the lesion.

12. The method according to claim 10 wherein the applicator is a swab.

13. The method according to claim 10 wherein the short or intermediate acting local anesthetics are selected from the group consisting of benzocaine, chloroprocaine, hexylcaine, procaine, lidocaine, mepivacaine, hydroxyprocaine, oxybuprocaine, proparacaine and tetracaine.

14. The method according to claim 10 wherein the long acting local anesthetic is selected from the group consisting of Articaine, bupivacaine, cenchocaine, etidocaine, levobupivacaine, prilocaine, ropivacaine and trimecaine.

15. The method according to claim 10 wherein the short or intermediate acting anesthetic is lidocaine and the long acting local anesthetic is bupivacaine.

16. The method according to claim 15 wherein the anesthetics are present each from about 40% to 60% w/w as a percent of the anesthetics.
